(19) United States
(12) Patent Application Publication
(10) Pub. No.: US 2016/0278785 A1
(43) Pub. Date: Sep. 29, 2016

(54) BIOLOGIC-BASED EXPANDABLE OCCLUSION DEVICES

(71) Applicant: BOSTON SCIENTIFIC SCIMED, INC., Maple Grove, MN (US)

(72) Inventors: Joseph Michael Connolly, Minneapolis, MN (US); David Raab, Minneapolis, MN (US); Mark Smith, Coon Rapids, MN (US)

(21) Appl. No.: 15/083,263

(22) Filed: Mar. 28, 2016

Related U.S. Application Data
(60) Provisional application No. 62/138,511, filed on Mar. 26, 2015.

(51) Int. Cl. A61B 17/12; A61L 31/04

(52) U.S. Cl. CPC A61B 17/12131; A61B 17/12109; A61L 31/044; A61B 2017/1205; A61L 2430/36

(57) ABSTRACT

The present disclosure provides occlusion devices which comprise (a) an elongated core portion having a longitudinal axis and (b) a biologic layer comprising a hydrophilic natural polymer that expands upon contact with bodily fluid disposed on the elongated core portion. Also provided are assemblies and kits that contain such occlusion devices, and methods of delivering such occlusion devices to a patient.

### CROSS-REFERENCE TO RELATED APPLICATIONS

This application claims priority under 35 U.S.C. §119(e) to co-pending U.S. Provisional Application Ser. No. 62/138,511, filed on Mar. 26, 2015, herein incorporated by reference in its entirety.

### FIELD OF THE DISCLOSURE

This application relates generally to devices, assemblies and kits for creating occlusions in body lumens such as spermatic ducts, fallopian tubes, and blood vessels and to methods for creating occlusions using the same.

### BACKGROUND

The endovascular treatment of a variety of conditions throughout the body is an increasingly important form of therapy. Blood vessel occlusion devices are known which are placed within the vasculature of the body in order to form a physical barrier to blood flow and/or promote thrombus formation at the site.

The present disclosure pertains to improved devices, assemblies, kits and methods for occlusion of body lumens including blood vessels, among others.

### SUMMARY

In various aspects, biologic-based occlusion devices are provided which comprise the following: (a) an elongated core portion having a longitudinal axis and (b) a biologic layer comprising a hydrophilic natural polymer that expands upon contact with bodily fluid disposed on the elongated core portion.

In some embodiments, the hydrophilic natural polymer is collagen.

In some embodiments, which may be used with any of the above aspects and embodiments, the device may further comprise a layer of bioerodible material over the biologic layer.

In some embodiments, which may be used with any of the above aspects and embodiments, the biologic layer may expand in thickness by an amount ranging from 1.5 times to 15 times after immersion in normal saline at 37° C. for 1 hour.

In some embodiments, which may be used with any of the above aspects and embodiments, the elongated core portion may comprise one or more regions of reduced diameter, and the hydrophilic natural polymer may be disposed in the one or more regions of reduced diameter.

In some embodiments, which may be used with any of the above aspects and embodiments, the elongated core portion may be in the form of a solid rod.

In some embodiments, which may be used with any of the above aspects and embodiments, the elongated core portion may be in the form of a tubular member (e.g., in the form of a tube with a solid wall or in the form of a helically wound wire, etc.).

In some embodiments, which may be used with any of the above aspects and embodiments, the elongated core portion may have an unconstrained memorized shape that is in the form of a three-dimensional spiral (e.g. a helix, a conic spiral, etc.).

In some embodiments, which may be used with any of the above aspects and embodiments, the occlusion device may further comprise an anchoring element attached to the elongate core portion. For instance, the elongate core portion may comprise a plurality of barbs or tines as anchoring elements, or the anchoring element may have an unconstrained memorized shape that is in the form of a three-dimensional spiral (e.g. a helix, a conic spiral, etc.), among other possibilities.

In some embodiments, which may be used with any of the above aspects and embodiments, the occlusion device may comprise an attachment feature.

Other aspects of the present disclosure pertain to assemblies, which may comprise (a) an occlusion device in accordance with any of the above aspects and embodiments and (b) an elongated delivery member that is configured to be attached to and detached from the occlusion device.

Other aspects of the present disclosure pertain to kits that comprise (a) an occlusion device in accordance with any of the above aspects and embodiments and (b) at least one article selected from (i) an elongate delivery member that is configured to be attached to and detached from the occlusion device, (ii) a catheter or sheath suitable for delivering the occlusion device to an occlusion site, or (iii) a catheter introducer.

In some embodiments, which may be used with any of the above aspects and embodiments, the occlusion device may be preloaded into a tubular device.

Other aspects of the present disclosure pertain to methods of treatment comprising introducing an occlusion device in accordance with any of the above aspects and embodiments into a body lumen (e.g., selected from spermatic ducts, fallopian tubes, normal blood vessels, and abnormal blood vessels, including aneurysms, arteriovenous fistulas, arteriovenous malformations, etc.), at which point the occlusion device is exposed to bodily fluid (e.g., blood, etc.), whereupon the occlusion device self-expands and impedes flow through the body lumen. In some embodiments, the body lumen is a blood vessel selected from the gastric arteries, the splenic artery, the gastroduodenal artery, and spermatic or ovarian veins.

The occlusion devices described herein may have a number of potential advantages including one or more of the following: (a) short occlusion times in the treatment of a variety of body lumens, (b) reduced distal migration, (c) reduced recanalization rates through increased tissue integration, (d) reduced procedural times and (e) reduced radiation dosages to patients (e.g., where x-ray fluoroscopy is employed).

These and other aspects, embodiments and advantages of the present disclosure will become immediately apparent to those of ordinary skill in the art upon review of the detailed description and claims to follow.

### BRIEF DESCRIPTION OF THE DRAWINGS

FIG. 1A is a schematic side view of a self-expanding occlusion device in accordance with an embodiment of the present disclosure;

FIG. 1B is a cross sectional view of the self-expanding occlusion device taken along section B-B of FIG. 1A;

FIG. 2A is a schematic side view of a self-expanding occlusion device of FIG. 1A after expansion upon exposure to bodily fluid in accordance with an embodiment of the present disclosure;

FIG. 2B is a cross sectional view of the self-expanding occlusion device taken along section B-B of FIG. 2A;

FIG. 2C is a schematic illustration of a portion of a helically wound wire, which may be useful in forming a body of a device like that of FIG. 1A, among others;

FIG. 3 is a schematic side view of a self-expanding occlusion device in accordance with another embodiment of the present disclosure;

FIG. 4 is a schematic side view of a self-expanding occlusion device in accordance with another embodiment of the present disclosure;

FIG. 5 is a schematic side view of a self-expanding occlusion device in accordance with still another embodiment of the present disclosure;

FIG. 6 is a schematic side view of a self-expanding occlusion device in accordance with yet another embodiment of the present disclosure; and

FIGS. 7A-7D are schematic partial cross-sectional views showing the deployment in a body lumen of an occlusion device in accordance with an embodiment of the present disclosure.

Unless otherwise provided in the following specification, the drawings are not necessarily to scale, with emphasis being placed on illustration of the principles of the disclosure.

### DETAILED DESCRIPTION

A more complete understanding of the present disclosure is available by reference to the following detailed description of numerous aspects and embodiments of the disclosure. The detailed description which follows is intended to illustrate but not limit the disclosure.

The terms “proximal” and “distal” generally refer to the relative position, orientation, or direction of an element or action, from the perspective of a clinician using the medical device, relative to one another. Thus, “proximal” may generally be considered closer to the clinician or an exterior of a patient, and “distal” may generally be considered to be farther away from the clinician, along the length or beyond the end of the medical device.

The present disclosure pertains to devices, assemblies and kits for creating occlusions in body lumen occlusions including, for example, spermatic ducts, fallopian tubes, normal blood vessels, and abnormal blood vessels, including aneurysms, arteriovenous fistulas and arteriovenous malformations, among others. The occlusion devices of the present disclosure may be configured to fit within a tubular device such as a catheter or delivery sheath for delivery to a desired delivery site in a body lumen. Upon exiting the tubular device the occlusive device is exposed to bodily fluid, causing the occlusive device to swell and naturally expand to an expanded configuration, thereby at least partially occluding the body lumen. For example, in certain embodiments pertaining to blood vessels, the occlusion devices may be pushed from the distal end of a catheter that is in place at the site of embolization. Upon exiting the catheter, the device will contact blood and expand within the blood vessel, at least partially occluding the same.

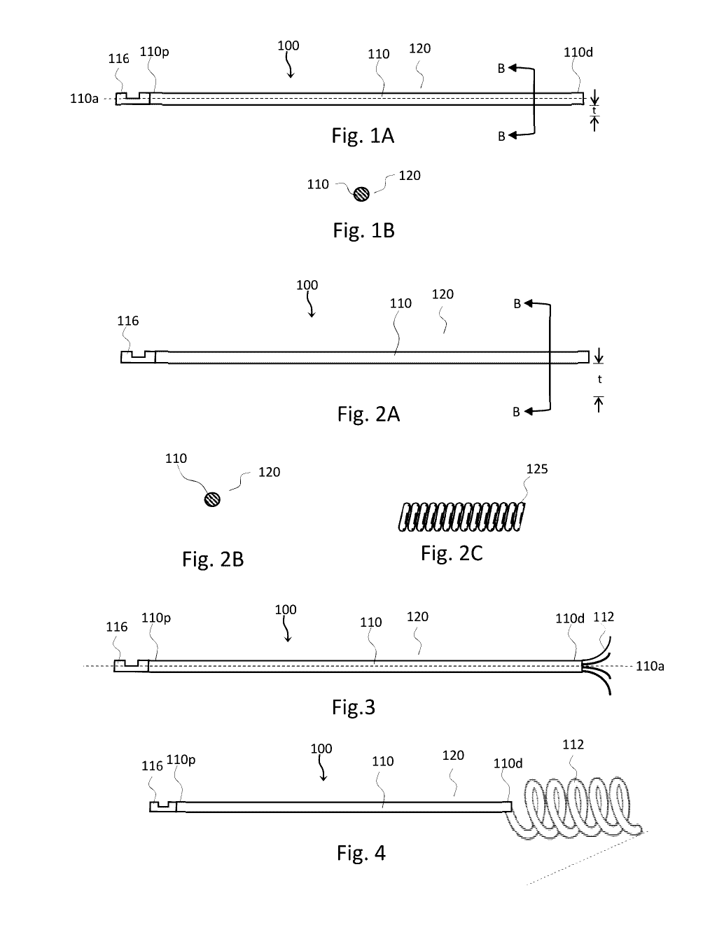

FIG. 1A is a schematic view of a self-expanding occlusion device 100 in a contracted state, in accordance with an embodiment of the present disclosure. FIG. 1B is a schematic view of the self-expanding occlusion device 100 of FIG. 1A taken along section B-B of FIG. 1A.

As seen from these FIGS. 1A-1B, the occlusion device 100 is generally cylindrical in shape and comprises a core portion 110 having a proximal end 110p, a distal end 110d, and an axis 110a, at least partially covered by a biologic layer 120 having a thickness t. The core portion 110 is preferably an elongated core portion 110 in that it has a length-to-width ratio ranging from 2.1 up to 1600:1 or more, for example, ranging from 2:1 to 5:1 to 10:1 to 25:1 to 50:1 to 100:1 to 250:1 to 500:1 to 1000:1 to 1600:1 (i.e., ranging between any two of the preceding values). The occlusion device 100 in the embodiment shown is provided with an optional attachment feature 116 for attachment to and detachment from a delivery member as discussed further below. Where present, the attachment feature 116 may be of a unitary structure with the core portion 110, or the attachment feature 116 may be provided in the form of a separate component that is attached to the core portion 110, for example, soldered, welded, fused, glued, crimped, or otherwise joined together with the core portion 110.

In the embodiment shown, the biologic layer 120 covers the entirety of the core portion 110 except for the proximal end 110p and distal end 110d of the device. The biologic layer 120 may cover more or less of the core portion 110 than is shown, including the coverage of particular regions of the core portion 110 as discussed in more detail below.

The biologic layer 120 is configured to expand in a body lumen (e.g., in a blood vessel), thereby assisting the device in slowing or immediately halting flow through the body lumen (e.g., blood flow) upon expansion. Expansion of the biologic layer 120 occurs as a result of swelling of the biologic layer 120 upon absorption of aqueous bodily fluid (e.g., blood). In this regard, FIG. 2A is a schematic view of the self-expanding occlusion device 100 of FIG. 1A, after exposure to bodily fluid, showing a significant increase in the thickness t of the biologic layer 120. FIG. 2B is a schematic view of the occlusion device 100 of FIG. 2A taken along section B-B of FIG. 2A.

In certain embodiments, the biologic layer may expand after exposure to aqueous fluid to a thickness t that is from 1.5 to 3 to 5 to 10 to 15 or more times the thickness t of the biologic layer (i.e., ranging between any two of the preceding values) prior to exposure to aqueous fluid. The ability of a given biologic layer to expand upon exposure to aqueous fluid can be measured by comparing the thickness of the biologic layer in a dry state (e.g., its state when packaged in suitable medical device packaging) to the thickness of the biologic layer after immersion in normal saline at 37° for 1 hour. In certain embodiments, the biologic layer 120 may constitute between 10% and 70% of the overall width (i.e., diameter) of the device.

Typical thicknesses for the biologic layers of the devices of the present disclosure may vary considerably, typically ranging, for example, from between 0.002″ (0.05 mm) at 0.025″ (0.64 mm) in thickness, among other values, depending on catheter size and application.

Beneficial biological materials for forming the biologic layer include hydrophilic natural polymers such as peptides (defined herein as amino-acid polymers containing from 2 to 50 amino acids) and proteins (defined herein as amino-acid polymers containing more than 50 amino acids), as well as polysaccharides. Specific examples of biologic materials include, for example, collagen, gelatin, fibrin, albumin, hyaluronic acid, glycosaminoglycans, alginates (including alginic acid and its derivatives), agarose, chitosan, cellulosic polymers such as carboxymethyl cellulose, starches including hydroxyethyl starch and dextran polymers including dextran and carboxymethyl dextran, among others.

A particularly beneficial hydrophilic natural polymer may be, for example, collagen, more than 20 types of which have been identified. Collagens have a triple helix motif composed of three chains, each of which comprises an amino acid sequence in which glycine (Gly) is typically present as every third residue (e.g., Gly-X-Y, where X and Y are variable and are often Pro or Hyp (hydroxyproline)). In certain embodiments, the biologic layer in the devices of the present disclosure may comprise type I collagen, type III collagen, or a mixture of type I collagen and type III collagen, among other collagen types.

In certain embodiments the biologic layer may comprise a hydrophilic natural polymer blended with, or covalently attached, to a hydrophilic synthetic polymer. Suitable hydrophilic synthetic polymers may be selected, for example, from polyethers including polyalkylene oxides such as polyethylene oxide (also referred to as polyethylene glycol), polypropylene oxide and polyethylene oxide-polypropylene oxide copolymers, polyols such as polyvinyl alcohol, polyacids such as polyacrylic acid, polymethacrylic acid and derivatives thereof, including polyacrylates, polymethacrylates such as poly(2-hydroxyethyl methacrylate) (which is also a polyol) and polyacrylamides including poly(N-isopropylacrylamide), polyamines, hydrolyzed polyacrylonitrile, poly(vinyl pyrrolidone), polyphosphazene, hydrophilic polyurethanes and synthetic hydrophilic polypeptides (e.g., polymers and copolymers of hydrophilic amino acids such as arginine, lysine, asparagine, glutamic acid, aspartic acid, and proline), among others.

In certain embodiments, the hydrophilic natural polymer in the biologic layer may be covalently crosslinked to enhance stability of the biologic layer. Alternatively or in addition, the hydrophilic synthetic polymer (where present) may be covalently crosslinked to enhance stability of the biologic layer.

Prior to delivery to a body lumen (e.g., a blood vessel, etc.) the biologic layer 120 is preferably maintained in a substantially non-hydrated contracted configuration, for example, as shown in the embodiment of FIGS. 1A-1B. Upon contact with a bodily fluid (e.g., blood, etc.), the biologic layer 120 expands as shown schematically in FIGS. 2A-2B, thereby taking up more space in the body lumen. The expanded biologic layer 120 may cause a physical slowing of the flow of bodily fluid as discussed in more detail below.

Where the bodily fluid is blood, the expanded biologic layer 120 may also act as a substrate for platelet adhesion. For example, it is well known that collagen is highly thrombogenic, leading to quick clot formation. Collagen is also known to act as a substrate for fibroblasts and to enhance tissue ingrowth, which is desirable from a vessel occlusion standpoint as tissue ingrowth acts as an obstacle to recanalization.

The core portion 110 of the occlusion device 100 may be formed of, or comprise, a metallic material, a ceramic material, a polymeric material, a metallic-polymeric composite material, a ceramic-polymeric composite material, or a metallic-ceramic composite material, among others. Some specific examples of suitable materials may be selected from metals and metal alloys such as platinum, nickel, titanium nickel-titanium alloy (nitinol) (e.g., super elastic or linear elastic nitinol), stainless steel (e.g., 303, 304v, or 316L stainless steel), nickel-chromium alloy, nickel-chromium-iron alloy, and cobalt alloy, as well as synthetic and natural polymers, for example, polyamides such as nylon, polyesters such as polyethylene terephthalate (e.g., DACRON), and fluoropolymers such as polytetrafluoroethylene (PTFE) (e.g., expanded PTFE), among others.

In various embodiments, the attachment feature 116 of the occlusion device 100 may also be formed of, or comprise, a metallic material, a ceramic material, a polymeric material, a metallic-polymeric composite material, a ceramic-polymeric composite material, or a metallic-ceramic composite material, among others. Some specific examples of suitable materials may be selected from those listed above. In certain embodiments, the attachment feature 116 may be formed from the same material as the core portion 110 of the occlusion device 100.

The material selected for the core portion 110 may be soft or hard, depending on the specific condition being treated. For example, a soft material such as a polymeric material may be employed where aneurysms are being treated, whereas a hard material such as a hard metallic material may be used in high flow vessels such as pulmonary arteriovenous malformations. In some embodiments, the core portion 110 may have a transition in hardness along the axial length (e.g., being harder at the distal end for better anchoring and softer in regions where the hydrophilic natural polymer is applied. Changes in hardness and softness can also be implemented by changing coil wind in some embodiments.

In various embodiments, the occlusion device 100 may include imaging markers (not shown), for example, radiopaque markers, which may be positioned at various points along the device 100, so as to provide information on the position and/or orientation of the device 100. Radiopaque materials may be, for example, attached, electroplated, dipped and/or coated at one or more locations along the core portion 110 of the device. Radiopaque materials may be, for example, dispersed within the material forming the biologic layer 120 of the device. Radiopaque materials are materials capable of being detected on a fluoroscopy screen or another imaging technique such as X-ray during a medical procedure. Suitable radiopaque materials may include metals such as gold, platinum, palladium, tantalum, tungsten, metal alloys comprising one or more of the preceding metals, barium sulfate, bismuth subcarbonate, iodine and iodine-containing materials, among others. In certain beneficial embodiments, radiopaque markers (e.g., in the form of a band) may be positioned at the distal and proximal ends of the device, also possibly with markers along the length of the device.

A number of methods exist for attaching the biologic layer 120 to the core portion 110. For example, biologic layer 120 may be secured to the core portion 110 through the use of biocompatible staples, sutures or combinations thereof. In some embodiments, the core portion 110 may include a plurality of barbs or other anchors, which may project through the biologic layer 120, holding it in place.

The biologic layer 120 may be secured to the core portion 110 by adhesive bonding wherein the biologic layer 120 is bonded to itself and/or to the core portion 110. Examples of adhesives include, for example, cyanoacrylate adhesives, urethane adhesives, and UV adhesives, among others. In some embodiments, the core portion 110 may be modified to increase surface area in order to increase adhesion, for example, using a suitable technique such as surface etching.

In some embodiments, a filamentous material 114, such as a wire, may be wound around the biologic layer 120 to hold it in place, for example, as shown in FIG. 6. In some embodiments, circumferential clips (not shown) may be placed around the biologic layer 120 in multiple spots to ensure attachment. The circumferential clips may also be used as radiographic marker bands.

In some embodiments, the biologic layer 120 may be initially constructed in the form of a hollow annular member into which the core portion 110 is inserted. For instance, collagen molding techniques have advanced to the point where complex geometries can be produced. See, e.g., A J Reiffel et al. (2013) “High-Fidelity Tissue Engineering of Patient-Specific Auricles for Reconstruction of Pediatric Microtia and Other Auricular Deformities”. PLoS ONE 8(2): e56506. doi:10.1371/journal.pone.0056506, in which CAD/CAM techniques were used to develop molds for collagen type I hydrogel scaffolds and that precisely mimic the normal anatomy of the external ear. Using these and other techniques, collagen and other hydrophilic natural polymers may be molded into a wide variety of desired shapes.

In some embodiments, the core portion 110 may be in the form of a rod. The rod may be, for example, in the form of a solid rod or in the form of a hollow rod, which may be, for instance, in the form of a helically wound wire 125 (see, e.g., FIG. 2C) or in the form of a hollow tube, which may be provided with a number of slots to increase flexibility. Typical rod diameters may range, for example, from 0.005″ (0.13 mm) to 0.030″ (0.76 mm), among other possibilities.

In certain embodiments, the core portion 110 may comprise a rod that is substantially linear when in an unconstrained state. In these embodiments, the occlusion device 100 may be sized and selected to expand to the insider diameter of the body lumen into which it is implanted (see, e.g., FIGS. 7A-7D, discussed below).

In certain embodiments the core portion 110 is provided with a shape memory. For example, the core portion 110 may comprise a hollow or solid rod which can assume a substantially linear shape when stretched for placement through the lumen of a delivery catheter, but which takes on a substantially non-linear memorized shape when in an unconstrained state. Examples of unconstrained shapes include various three-dimensional spiral shapes, including cone-shaped spirals, dual-cone-shaped spirals and cylinder-shaped spirals (i.e., helices), among others. Such a core portion 110 may be at least partially covered by a biologic layer 120 as described elsewhere herein.

In certain of these embodiments, the core portion 110 may comprise a coil analogous to embolic coils, which are commonly used for variety of medical procedures. For example, the core portion 110 may take the form of a coil formed from helically wound wire 125 such as that shown in FIG. 2C. Such a helically wound wire (which is in the form of a hollow rod) may be formed by winding a metallic (e.g., platinum, platinum alloy, etc.) wire strand about a first mandrel in the form of a rod. The relative stiffness of the coil depends, among other things, on its composition, the diameter of the wire strand, the diameter of the primary mandrel, and the pitch of the primary windings. The helically wound wire may then wrapped around a larger, secondary mandrel, and heated to impart a secondary shape. A coil having this type of construction may be used as a core portion 110 in an occlusion device 100 in accordance with the present disclosure. Upon delivery, the coil will attempt to achieve its secondary shape (i.e., its unconstrained shape), helping to anchor the occlusion device 100 in the body lumen. Potential secondary shapes for the coils include various three-dimensional spiral shapes, such as cone-shaped coils, dual-cone-shaped (also referred to as diamond-shaped) coils, and cylinder-shaped (helical) coils, among others.

In certain embodiments, the occlusion device 100 may be provided with one or more anchoring features such that the device may be better able to engage surrounding tissue and resist migration after implantation in a body lumen. For example, the occlusion device 100 may include a plurality of anchors (e.g., tines, barbs, hooks, etc.) extending radially outward from the core portion such that they can engage tissue and inhibit longitudinal movement of the deployed occlusion device.

For instance, turning to FIG. 3, there is shown therein an occlusion device 100 comprising an elongated core portion 110 (having a proximal end 110p, a distal end 110d and an axis 110a) at least partially covered by a biologic layer 120. The occlusion device 100 in the embodiment shown further includes an optional attachment feature 116 as previously described. Radiating from the distal end 110d of the elongated core portion are a plurality of tines 112, which may be formed, for example, from a material that has a shape memory such that the tines 112 are able to be compressed into a tube (not shown) and self-expand upon being removed from the tube. Some specific examples of suitable shape memory materials are set forth above and include metallic materials and/or alloys such as nickel-titanium alloy (nitinol) (e.g., super elastic or linear elastic nitinol), stainless steel (e.g., 303, 304v, or 316L stainless steel), nickel-chromium alloy, nickel-chromium-iron alloy, cobalt alloy, nickel, titanium, platinum, and the like. Because the tines 112 radiate outwardly as one moves along the axis 110a in a proximal-to-distal direction, the tines may be re-sheathed within a delivery tube (not shown) by withdrawing the occlusion device 100 in a proximal direction relative to the delivery tube. The tines 112 may be, for example, attached to the core portion 110 after formation of the core portion 110, or the tines 112 may be integrally formed with the core portion 110.

In certain embodiments, the anchoring feature may be in the form of a solid or hollow rod having a memorized shape that is configured to engage sidewalls of a body lumen into which the occlusion device is implanted. For example, the anchoring feature may comprise a solid or hollow rod (e.g., a helically wound wire or hollow tube, which may be provided with a number of slots to increase flexibility), which can assume a substantially linear shape when stretched for placement through the lumen of a delivery catheter, but which takes on a substantially non-linear memorized shape when in an unconstrained state. Examples of unconstrained shapes include various three-dimensional spiral shapes, including cone-shaped spirals, dual-cone-shaped spirals and cylinder-shaped spirals (i.e., helices), among others. In some embodiments, the anchoring feature may be uncoated. In some embodiments, at least outer tissue-engaging surfaces of the anchoring feature may be roughened to better engage surrounding tissue in some embodiments.

Turning to FIG. 4, there is shown a specific embodiment of an occlusion device 100 comprising an elongated core portion 110 having a proximal end 110p, a distal end 110d, at least partially covered by a biologic layer 120. The occlusion device 100 in the embodiment shown further includes an optional attachment feature 116, as previously described. The occlusion device 100 also further includes an anchoring feature in the form of a cylinder-shaped (helical) element 112. The helical element 112 may be formed from, for example, a solid or hollow rod that has a memorized unconstrained shape in the form of a helix, such that the helical element 112 is able to be compressed into a delivery tube (not shown) and self-expand upon being removed from the tube. The helical element 112 may also be re-sheathed within the delivery tube by withdrawing the occlusion device 100 in a proximal direction relative to the delivery tube. The helical element 112 may be formed, for example, from a material that has a shape memory, such as those described above. The helical element 12 may be attached to the core portion 110 after formation of the core portion 110, or the helical element 112 may be integrally formed with the core portion 110.

Anchoring elements 112 may be provided at the proximal end, the distal end, or both the proximal end and the distal end of the occlusion device 100.

As previously noted, occlusion devices in accordance with the present disclosure may be configured for delivery through catheters of various sizes. To reduce detrimental effects associated with catheter delivery, the occlusion devices may be, for example, delivered through the catheter while disposed within a sheath, thereby minimizing the amount of hydrophilic natural polymer lost due to friction when pushing the occlusion devices through the catheter. Examples of sheath materials include low friction polymers including fluoropolymers such as polytetrafluoroethylene (PTFE), among others. Once the occlusion device and sheath are delivered out of the catheter at a delivery site, the sheath can be retracted, exposing the hydrophilic natural polymer to bodily fluid (e.g., blood), causing expansion of the biologic layer.

In some embodiments, the hydrophilic natural polymer may be coated with a layer of low friction material that dissolves rapidly in biological fluid once the occlusion device is delivered into the body. For example, the biologic layer may be provided with a coating of a bioerodible material such as sugars (e.g., sucrose), water soluble gelatins, partially water soluble lipids such as triglycerides or phospholipids, or biodegradable polymers such as PLGA to improve pushability of the occlusion device, which bioerode upon contacting bodily fluids.

In some embodiments, a flushing solution that does not cause the not cause the hydrophilic natural polymer to swell, for example, a non-aqueous liquid, may be introduced into the catheter prior to pushing the occlusion device through the catheter.

Another strategy for reducing friction is to provide the core portion with a single section, or multiple repeating sections, of necked-down areas with smaller diameters where the hydrophilic natural polymer is situated. By ensuring that the total diameter of the occlusion device in the necked-down areas (core portion+hydrophilic natural polymer) is equal to or smaller than the diameter in the uncoated areas, friction between the hydrophilic natural polymer and delivery catheter may be reduced, reducing wear and/or improving pushability of the occlusion device.

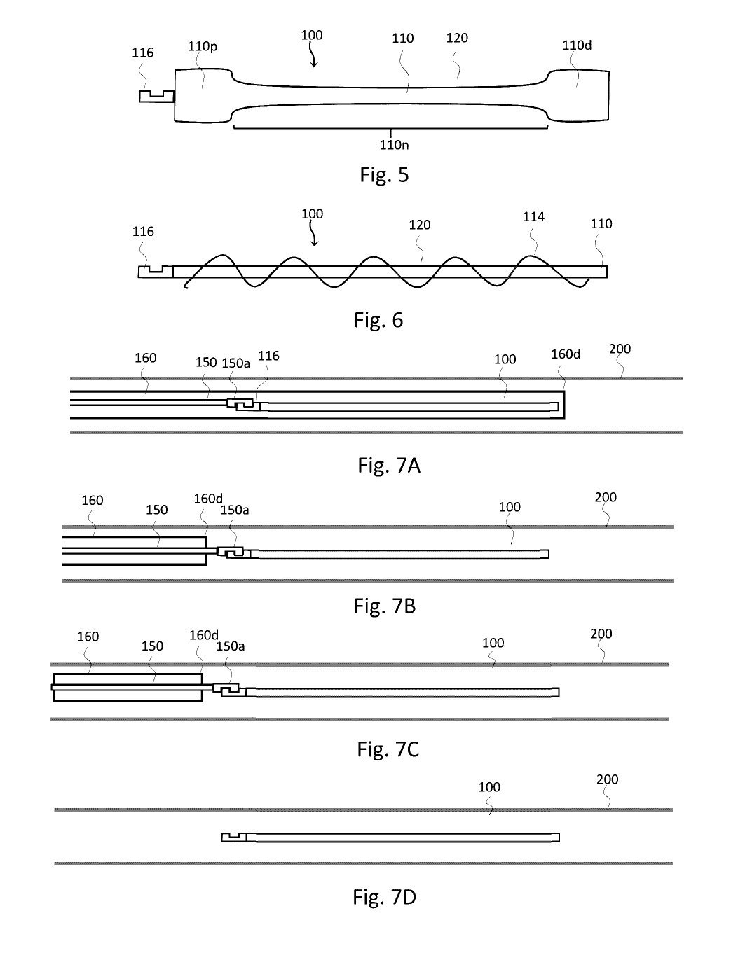

One example of such a device is shown in FIG. 5, where there is shown therein an occlusion device 100 comprising an elongated core portion 110 having a proximal end 110p and a distal end 110d. The core portion 110 is covered by a biologic layer 120 in a necked-down region 110n between the proximal end 110p and distal end 110d of the core portion 110. The core portion 110 in the embodiment shown further includes an optional attachment feature 116 as previously described. Because the overall diameter of the occlusion device 100 is smaller in the necked-down region 100n than in other portions of the occlusion device 100, frictional effects on the biologic layer 120 are minimized.

In alternative embodiments, a tube may be machined to provide hollow depressions, slots and/or holes within which the hydrophilic natural polymer may be placed in order to reduce friction between the hydrophilic natural polymer and delivery catheter. Made to provide hollow depressions, slots or holes that the hydrophilic natural polymer can be placed in in order to reduce friction between the hydrophilic coating and the delivery catheter.

As previously indicated, in various embodiments, the occlusion device 100 may have an expanded diameter. In various embodiments, an occlusion device is selected for implantation in a vessel that has a diameter that is, for example, up to about 10 times greater than a contracted diameter of the occlusion device, among other values.

In various embodiments, a vessel occlusion device having a compressed diameter sufficiently small to occupy a 0.021 inch inner diameter catheter (i.e., less than 0.021 inch or 0.53 mm) may have an expanded diameter after blood exposure that ranges, for example, from 1 mm to 5 mm, among other values. Such devices are, for instance, suitable for implantation in of a vessel having an inner diameter ranging, for example, from 1 mm to 5 mm, among other values.

In various embodiments, a vessel occlusion device having a compressed diameter sufficiently small to occupy a 0.027 inch inner diameter catheter (i.e., less than 0.027 inch or 0.69 mm) may have an expanded diameter after blood exposure that ranges, for example, from 2 mm to 7 mm, among other values. Such devices are, for example, suitable for implantation in a vessel having an inner diameter ranging, for example, from 2 mm to 7 mm, among other values.

In various embodiments, a vessel occlusion device having a compressed diameter sufficiently small to occupy a 5 French guide sheath having an inner diameter of approximately 0.067″ (i.e., 1.67 mm) or a 6 French guiding catheter having an inner diameter of approximately 0.070″ (1.8 mm) may have an expanded diameter after blood exposure that ranges, for example, from 5 mm to 14 mm, among other values. Such devices are, for example, suitable for embolization of a vessel having an inner diameter ranging, for example, from 5 mm to 14 mm, among other values.

With regard to length of the occlusion devices described herein, it is noted that this length can be designed to be longer or shorter and is not dictated by catheter dimensions. Device length is thus general design variable that can be adjusted as needed. In general, increasing the length of the occlusion device will increase the resistance of the device to migration within a body lumen. In this regard, more length may be advantageous when prevention of both antegrade and retrograde flow is desired, such as in the treatment of bleeds or aneurysms of the viscera, among other applications. It is further noted that a health practitioner may trim some of the length from the device in order to tailor the length of the device to the implant location.

In various embodiments, occlusion devices are provided in conjunction with a delivery system that includes an elongate delivery member and tubular delivery device.

With reference to FIGS. 7A-7D, for delivery, the occlusion device 100 may be introduced through a lumen of a tubular device such as a delivery catheter 160 having an open distal end 160d. An elongate delivery member such as a delivery shaft 150 may also be disposed within the lumen of the delivery catheter 160 and may be reversibly connected to the occlusion device 100 at the proximal attachment feature 116, such that the occlusion device 100 can be advanced and withdrawn relative to the catheter 160 as desired and eventually released within the body.

In some embodiments, the delivery shaft 150 may comprise an attachment mechanism 150a that is configured to reversibly engage an attachment feature 116 of the implantable device 110. For example, the attachment mechanism 150a may be complementary in shape to the attachment feature 116 as shown in FIGS. 7A-7C. While in the delivery catheter 160, these elements 152,116 are constrained in a clasped configuration like that shown, preventing them from disengaging. Once elements are 152,116 emerge from a distal end of the catheter, they may be readily separated, for example, by rotating the delivery shaft 150. As another example, the delivery shaft 150 may comprise a threaded male fitting at its distal end which is threaded into a female threaded fitting within the attachment feature of the occlusion device 100, or vice versa, among various other possible mechanical or electrical (e.g., electrolytic dissolution, etc.) means of forming such a reversible connection. The delivery catheter 160, occlusion device 100, and delivery shaft 150 collectively form a delivery system.

During delivery, the delivery system may be percutaneously inserted into a patient to deliver the occlusion device 100 to a desired vascular site 200 (e.g., an artery, vein, etc.). Access to an artery or vein to be embolized may be achieved via the femoral artery, femoral vein, or radial artery, among other access points. Initially, the occlusion device 100 is in a first, contracted configuration within the lumen of the delivery catheter 160, as shown in FIG. 7A.

Upon reaching the desired delivery location, the delivery catheter 160 may be withdrawn proximally while keeping the delivery shaft stationary, or the delivery shaft 150 may be advanced distally while the delivery catheter 160 is held stationary (i.e., relative movement between the delivery catheter and the delivery shaft 150 is created), such that the occlusion device 100 emerges from the delivery catheter 160 as shown in FIG. 7B. Depending on the nature of the linkage between the delivery shaft 150 and the occlusion device 100, the occlusion device 100 may be recaptured by pulling the occlusion device 100 back into the delivery catheter 160 (so long as the device has not expanded to a point where recapturing is no longer practical). Emergence from the delivery catheter 160 leads to contact between the occlusion device 100 and bodily fluid (e.g., blood), which results in swelling and thus radially outward expansion to an expanded configuration where the outer surface of the occlusion device 100 conforms to the wall of the vessel 200 as shown in FIG. 7C.

Lastly, delivery shaft 150 may be disconnected from the occlusion device 100 (if not previously released) and the delivery catheter 160 and delivery shaft 150 removed from the patient, leaving the occlusion device at the vascular site 200 as shown in FIG. 7D.

Once implanted, the biologic layer acts to slow or halt the blood flow, and the entire device may act as a substrate for coagulation and tissue ingrowth, creating a permanent embolus if desired.

Using these and other procedures, the occlusion devices described herein may be implanted in a variety of body lumens including spermatic ducts, fallopian tubes, and blood vessels. Where used for embolization, the devices described herein may be implanted into a wide variety of blood vessels, including a wide variety of arterial and venous blood vessels. Examples of arteries in which the devices may be implanted include following arteries (including any divisions thereof): the internal iliac artery (hypogastric artery), external iliac artery, gastroduodenal artery, renal artery, hepatic artery, uterine artery, lienal artery, splenic artery, intercostals artery, mesenteric artery, right gastric artery, left gastric artery, lumbar artery, internal carotid artery, communicating artery, basilar artery, bronchial artery, cerebral artery, cerebellar artery, profunda femoris artery, gastroepiploic artery, and pancreaticoduodenal artery, among others. Examples of veins in which the blood vessel embolization devices may be implanted include a pelvic vein, internal iliac vein (hypogastric vein), portal vein and gonadal veins (e.g. spermatic vein or ovarian vein, depending on gender), among others. Examples of blood vessels in which the blood vessel embolization devices may be implanted further include abnormal blood vessels, for example, arteriovenous fistulas and arteriovenous malformations, among others.

In particularly beneficial embodiments, occlusion devices as described herein may be employed to perform the following: varicocele and pelvic congestion syndrome (PCS) embolization (where many coils may be deployed to embolize a length of the vein from the gonads to the renal artery), aneurysms of the viscera that can be embolized via sandwich embolizations (proximal and distal) (e.g., by filling the aneurysm sac), bleeds of viscera (e.g., peptic ulcer bleeds of the gastroduodenal artery (GDA) or gastric artery, for instance by embolizing proximally and distally to the bleed to prevent antegrade and retrograde flow, respectively, wherein multiple devices may be employed), among others.

In another aspect of the disclosure, medical kits useful in embolization procedures are provided. The medical kits may include all or a subset of all the components useful for performing the procedures. For example, the medical kits may comprise any combination of any two, three, four, or more of the following items: (a) an occlusion device as described herein, (b) a tubular device (e.g., a catheter and/or sheath) suitable for delivering the vessel occlusion device (in certain beneficial embodiments, the vessel occlusion device preloaded into the tubular device), (c) an elongate delivery member such as a delivery shaft, which may be reversibly connected to the occlusion device via a suitable mechanism such as one of those described herein, (d) a catheter introducer, (f) suitable packaging material, and (g) printed material with one or more of the following: storage information and instructions regarding how to deploy the occlusion device in a subject.

Although various embodiments are specifically illustrated and described herein, it will be appreciated that modifications and variations of the present disclosure are covered by the above teachings and are within the purview of the appended claims without departing from the spirit and intended scope of the disclosure.

1. An occlusion device comprising: (a) an elongated core portion having a longitudinal axis and (b) a biologic layer comprising a hydrophilic natural polymer that expands upon contact with bodily fluid disposed on the elongated core portion.

2. The occlusion device of claim 1, wherein the hydrophilic natural polymer is collagen.

3. The occlusion device of claim 1, further comprising a layer of bioerodible material over the biologic layer.

4. The occlusion device of claim 1, wherein the biologic layer expands in thickness by an amount ranging from 1.5 times to 15 times after immersion in normal saline at 37° C. for 1 hour.

5. The occlusion device of claim 1, wherein the elongated core portion comprises one or more regions of reduced diameter and wherein the hydrophilic natural polymer is disposed in the one or more regions of reduced diameter.

6. The occlusion device of claim 1, wherein the elongated core portion is in the form of a solid rod.

7. The occlusion device of claim 1, wherein the elongated core portion is in the form of a tubular member.

8. The occlusion device of claim 7, wherein the tubular member is in the form of a helically wound wire.

9. The occlusion device of claim 1, wherein the elongated core portion has an unconstrained memorized shape that is in the form of a three-dimensional spiral.

10. The occlusion device of claim 9, wherein the three-dimensional spiral is selected from a helix and a conic spiral.

11. The occlusion device of claim 1, further comprising an anchoring element attached to the elongate core portion.

12. The occlusion device of claim 11, wherein the elongate core portion comprises a plurality of barbs or tines as anchoring elements.

13. The occlusion device of claim 11, wherein the anchoring element has an unconstrained memorized shape that is in the form of a three-dimensional spiral.

14. The occlusion device of claim 1, further comprising an attachment feature.

15. The occlusion device of claim 1, wherein the occlusion device is preloaded into a tubular device.

16. An assembly comprising the occlusion device of claim 1 and an elongated delivery member that is configured to be attached to and detached from the occlusion device.

17. The assembly of claim 16, wherein the occlusion device is preloaded into a tubular device.

18. A kit comprising:
(a) an occlusion device comprising: (i) an elongated core portion having a longitudinal axis and (ii) a biologic layer comprising a hydrophilic natural polymer that expands upon contact with bodily fluid disposed on the elongated core portion; and
(b) at least one article selected from (i) an elongate delivery member that is configured to be attached to and detached from the occlusion device, (ii) a catheter or sheath suitable for delivering the occlusion device to an occlusion site, or (iii) a catheter introducer.

19. A method of treatment comprising introducing an occlusion device into a blood vessel whereupon the occlusion device self-expands and impedes flow through the blood vessel, said occlusion device comprising: (a) an elongated core portion having a longitudinal axis and (b) a biologic layer comprising a hydrophilic natural polymer that expands upon contact with bodily fluid disposed on the elongated core portion.

20. The method of claim 19 wherein the body lumen is a blood vessel selected from gastric arteries, splenic artery, gastroduodenal artery, and spermatic or ovarian veins.
